(19) United States
(12) Patent Application Publication
(10) Pub. No.: US 2010/0016933 A1
(43) Pub. Date: Jan. 21, 2010

(54) COLD/HOT PACK

(75) Inventors: Yu-Gang Chen, Yongkang City (TW); Jung-Kuang Chen, Yongkang City (TW)

Correspondence Address: Wang Law Firm, Inc., 4989 Peachtree Parkway,, Suite 200, Norcross, GA, 30092, US

(73) Assignee: FAR EAST UNIVERSITY, Hsin-Shih (TW)

(21) Appl. No.: 12/501,470

(22) Filed: Jul. 12, 2009

(30) Foreign Application Priority Data
Jul. 15, 2008 (TW) 097126854

(51) Int. Cl. A61F 7/08; A61F 7/10

(52) U.S. Cl. 607/112

(57) ABSTRACT

A cold/hot pack includes a pack body and a binding section. The pack body includes a plurality of receiving spaces, and a liquid filled in each of the receiving spaces. The binding section functions to fasten and fix the pack body to a desired body area requiring cold or hot therapy.

### FIELD OF THE INVENTION

The present invention relates to a cold/hot pack, and more particularly to a cold/hot pack including a plurality of receiving spaces for containing a refrigerant or a heat-preserving compound therein in order to ensure therapeutic effect.

### BACKGROUND OF THE INVENTION

People pay more and more attention to their body health and therefore often take exercises outdoors. Sometimes, unexpected injury during exercises would occur, such as a sprained ankle, etc., and the injured area requires timely cold or hot therapy with a cold/hot pack. A conventional cold/hot pack usually has a bag-shaped body made of a plastic or a rubber material for holding ice cubes or hot water therein. The cold/hot pack is then positioned at the injured area. In the event the injured area is small in size and requires the cold/hot pack, the user has to hold down the cold/hot pack at the injured area with one or even two hands.

By doing this, the user is restricted from moving and doing other things freely 20 while using the cold/hot pack to soothe the injured area. Thus, the conventional large-size cold/hot pack is not so convenient for use.

Taiwan Patent Number M319745 discloses a fixable cold/hot pack device, which includes a pack body having a fixing section, and two fastening bands having different lengths. When a ratio of length of the two bands is between 2.5 and 3.5, the cold/hot pack device is particularly suitable for use at a user's neck portion; and when the ratio is between 3.5 and 6, the cold/hot pack device is particularly suitable for use at the user's forehead.

The above-mentioned Taiwan Patent also discloses another fixable cold/hot pack device, which includes a pack body having a first fixing section, and a band. When a ratio of the band length to a pack body width is between 0.6 and 2, the cold/hot pack device is particularly suitable for use at the user's arms and shanks, preventing the pack body from slipping away or deviating from the injured area. However, according to the disclosure of the above-mentioned Taiwan Patent Number M319745, the pack body is fixed to an injured position with a large area, and can only be used at some specific body areas limited by the ratio of the band lengths or of the band length to the pack body width. Therefore, the cold/hot pack device of Taiwan Patent Number M319745 is insufficient and impractical for use in terms of its usage to injured areas other than the above-mentioned specific body areas.

It is therefore tried by the inventor to develop an improved cold/hot pack to overcome the drawbacks in the conventional cold/hot pack device.

### SUMMARY OF THE INVENTION

A primary object of the present invention to provide a cold/hot pack that is more convenient for use in cold/hot therapy.

To achieve the above and other objects, the cold/hot pack according to the present invention includes a pack body and a binding section. The pack body includes a receiving space, and a liquid filled in the receiving spaces. The binding section is used to fasten and fix the pack body.

The cold/hot pack of the present invention provides the following advantages:
(1) The pack body includes a plurality of continuously connected receiving spaces, enabling the cold/hot pack to be conveniently used at both large joints and small knuckles.
(2) The pack body can be fixedly held to an injured body area by the binding section, allowing a user to move around freely while accepting cold/hot therapy with the cold/hot pack.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a schematic plan view of a cold/hot pack according to a preferred embodiment of the present invention; and

FIG. 2 shows an example of using the cold/hot pack of the present invention to soothe pains of an injured finger.

### DETAILED DESCRIPTION OF THE PREFERRED EMBODIMENTS

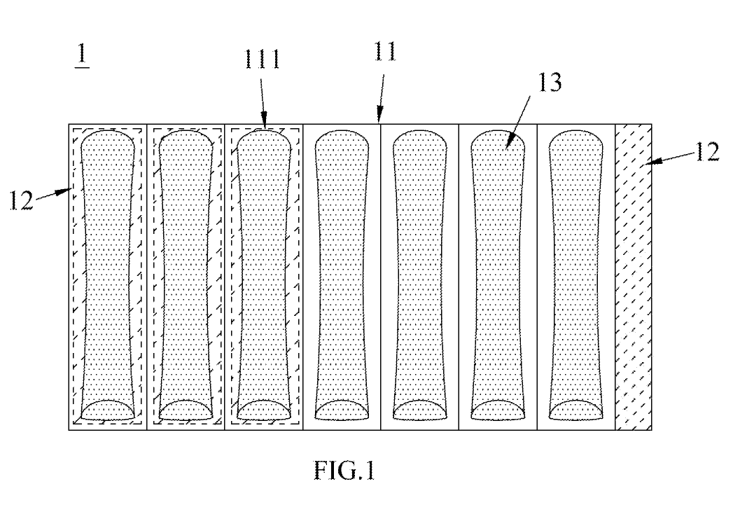

Please refer to FIG. 1, in which a cold/hot pack 1 according to a preferred embodiment of the present invention is shown. To enable easy understanding, the same elements in the illustrated embodiment are denoted by the same reference numeral. As shown in FIG. 1, the cold/hot pack 1 includes a pack body 11 and at least one binding section 12. The pack body 11 includes a plurality of parallel receiving spaces 1l, in each of which a liquid 13 is filled. The binding section 12 is provided on two sides of the pack body 11 for binding and fixing the pack body 11 to a desired area on a user's body. With the plurality of receiving spaces 111 provided on the pack body 11, the pack body 11 can be more conveniently wrapped around an injured area.

The pack body 11 is made of a waterproof material to prevent the liquid 13 filled in the receiving spaces 111 from leaking out of or permeating through the pack body 11. The material for making the pack body 11 can further be a heat-enduring material to particularly ensure the heat therapeutic effect of the cold/hot pack 1. The liquid 13 can be a refrigerant or a heat-preserving compound. A user may select a desirable cold pack or a hot pack for use according to actual need. The binding section 12 is preferably a fastening mechanism, such as, for example, a magic tape consisting of a loop tape and a hook tape, or buttons, or the like.

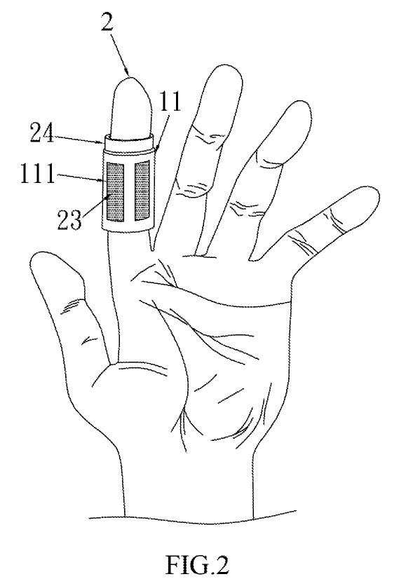

Please refer to FIG. 2 that shows an example of application of the cold/hot pack of the present invention to soothe pains of an injured finger. In the event a user's finger 2 is injured due to unexpected collision with something or sprain during exercise, the user can soothe the pains of the injured finger 2 by applying the cold/hot pack 1 having a refrigerant 23 filled in the receiving spaces 111 to the injured finger 2. To do so, the user can properly wrap the pack body 11 around the injured finger 2 according to the finger's thickness and the injury condition, and attach the binding section 12 (as shown FIG. 1) provided on two sides of the pack body 11 to each other, so that the pack body 11 is wrapped around and fixed to the injured finger 2. Wherein, the binding section 12 is a magic tape.

Since the cold/hot pack 1 comprises a plurality of continuously connected units, the pack body 11 can be properly adjusted in size corresponding to the location and/or the area of an injured portion to be covered with the pack body 11. For example, when a user's knee is injured, a relatively large-size cold/hot pack 1 may be used to wrap around the injured knee by one circle, so that the injured knee is fully covered by the pack body 11 without hindering the user from moving conveniently. Or, when a user's leg is injured, the injured leg can be covered with a large-size cold/hot pack 1 in the same manner as described above.

Most of the currently commercially available cold/hot packs are quite large in size for soothing the pains of a relatively large joint or a relatively wide muscular area. These large-size cold/hot packs are not convenient for use in terms of a small joint, such as a finger knuckle, or a non-planar injured area that requires cold therapy or hot therapy. With the cold/hot pack 1 of the present invention, even an injured body area at the small finger knuckle, for example, can be conveniently wrapped with the pack body 11 and the binding section 12 to achieve the purpose of cold or hot therapy. Further more, since the cold/hot pack 1 of the present invention can be directly fixed to the user's finger, the user may still freely move around without being confined during the cold or hot therapy with the cold/hot pack 1. The cold/hot pack comprises a shaped seat 24 to be a shaped auxiliary tool. When a refrigerant compound filled in the receiving spaces 111, the pack body 11 can fix on the shaped seat by the binding section. 12. Then put the pack body 11 in the refrigerator for congealing the refrigerant compound. The shaped seat 24 is a columnar shape for covering the injured fingers and reducing the discomfort of the injured fingers.

The present invention has been described with a preferred embodiment thereof and it is understood that many changes and modifications in the described embodiment can be carried out without departing from the scope and the spirit of the invention that is intended to be limited only by the appended claims.

What is claimed is:

1. A cold/hot pack, comprising:
a pack body including a plurality of parallel receiving spaces, and a liquid filled in each of the receiving spaces; and
a binding section arranged for binding and fixing the pack body to a desired area.

2. The cold/hot pack as claimed in claim 1, wherein the desired area is a finger or a knee.

3. The cold/hot pack as claimed in claim 1, wherein the pack body is made of a waterproof material.

4. The cold/hot pack as claimed in claim 1, wherein the pack body is made of a heat-enduring material.

5. The cold/hot pack as claimed in claim 1, wherein the cold/hot pack comprises a shaped seat to be a shaped auxiliary tool.

6. The cold/hot pack as claimed in claim 1, wherein the binding section comprises a plurality of fastening mechanism.

7. The cold/hot pack as claimed in claim 6, wherein the fastening mechanism is provided on two sides of the pack body for fastening and fixing the pack body in place.

8. The cold/hot pack as claimed in claim 6, wherein the fastening mechanism can be a magic tape structure consisting of a loop tape and a hook tape.

9. The cold/hot pack as claimed in claim 1, wherein the liquid can be a refrigerant or a heat-preserving compound.
